SEQUENCE LISTING (8 sequences)

SEQ ID NO: 1 - DNA, 25 bp, synthetic construct
  misc_feature 1..25: note = sgRNA1 upstream primer
  source 1..25: mol_type = other DNA
caccgaataggaatttgtgacgtcc

SEQ ID NO: 2 - DNA, 25 bp, synthetic construct
  misc_feature 1..25: note = sgRNA1 downstream primer
  source 1..25: mol_type = other DNA
aaacggacgtcacaaattcctattc

SEQ ID NO: 3 - DNA, 21 bp, synthetic construct
  misc_feature 1..21: note = sgRNA1
  source 1..21: mol_type = other DNA
gaataggaatttgtgacgtcc

SEQ ID NO: 4 - DNA, 25 bp, synthetic construct
  misc_feature 1..25: note = sgRNA2 upstream primer
  source 1..25: mol_type = other DNA
caccgagctcagcgcaatctttatc

SEQ ID NO: 5 - DNA, 24 bp, synthetic construct
  misc_feature 1..24: note = sgRNA2 downstream primer
  source 1..24: mol_type = other DNA
aaacgataaagattgcgctgagct

SEQ ID NO: 6 - DNA, 21 bp, synthetic construct
  misc_feature 1..21: note = sgRNA2
  source 1..21: mol_type = other DNA
gagctcagcgcaatctttatc

SEQ ID NO: 7 - DNA, 20 bp, synthetic construct
  misc_feature 1..20: note = Zfp445-Forward
  source 1..20: mol_type = other DNA
agtgcgtccttcgttacctg

SEQ ID NO: 8 - DNA, 20 bp, synthetic construct
  misc_feature 1..20: note = Zfp445-Reverse
  source 1..20: mol_type = other DNA
gtgaaggtagctggggatac

(19) United States
(12) Patent Application Publication
(10) Pub. No.: US 2023/0295618 A1
(43) Pub. Date: Sep. 21, 2023

(54) PREPARATION METHOD FOR MUTANT CELLS WITH DELETION OF TARGET GENES

(71) Applicant: ShanghaiTech University, Shanghai (CN)

(72) Inventor: Xiajun LI, Shanghai (CN)

(73) Assignee: ShanghaiTech University, Shanghai (CN)

(21) Appl. No.: 18/181,583

(22) Filed: Mar. 10, 2023

(30) Foreign Application Priority Data
Mar. 21, 2022 (CN) 2022103045795

(51) Int. Cl. C12N 15/11; C12N 15/85; C12N 5/0735

(52) U.S. Cl. CPC C12N 15/111; C12N 15/85; C12N 5/0606; C12N 2510/00

(57) ABSTRACT

A preparation method for mutant cells with deletion of target genes, at least comprising the following steps: (1) transfecting a sgRNA group into same host cells, where the sgRNA group includes two sgRNAs that target different sites of a same target gene, and the number of the sgRNA group may be one or more than one. When the number of the sgRNA group is more than one, each different sgRNA group targets one different target gene. (2) Enriching the host cells expressing one or more sgRNA groups. (3) Culturing the host cells obtained from step (2) to produce mutant cells with deletion of target genes.

### FIELD OF THE INVENTION

The present disclosure relates to the field of gene editing technology, particularly to a preparation method for mutant cells with deletion of target genes.

### BACKGROUND OF THE INVENTION

Gene editing, which is also called genome editing or genome engineering, is a new technology for modifying a target gene in the genome of many different living organisms or cells. It enables the splicing of the target gene in the genome through a site-specific endoribonuclease and results in the cleavage of double-stranded DNA. Then a self-repair pathway is activated to cause an insertion, a deletion, or a replacement of the bases at the target sites, which results in genetic mutations at the target sites in the genome.

Gene editing technology can generate point mutations in the cells with high efficiency. However, the efficiency is relatively low for the reported gene editing with two sgRNAs that are used to generate mutant cells with deletion of target genes, such as deletion mutations in the embryonic stem (ES) cells.

### SUMMARY OF THE INVENTION

Since there are some difficulties in obtaining mutant cells with deletions according to the previous methods, we have developed a novel and more effective method for generating mutant cells with deletions.

In the first aspect, the present disclosure provides a preparation method for mutant cells with deletion of target genes, at least including the following:
step (1) transfecting a sgRNA group into same host cells, where the sgRNA group includes two sgRNAs that target two different sites of a same target gene, and the number of the sgRNA group can be one or more than one. When the number of the sgRNA group is more than one, each different sgRNA group targets one different target gene.
Step (2) enriching the host cells expressing one or more sgRNA groups.
Step (3) culturing the host cells obtained from step (2) to produce mutant cells with deletion of target genes.

In the second aspect, the present disclosure provides mutant cells with deletion of target genes, which is produced according to the preparation method mentioned above.

The preparation method for mutant cells with deletion of target genes has the following advantages:

The transfection followed by drug selection within a short period of time leads to enrichment of the transfected cells containing one or more sgRNA expression vectors, which results in highly efficient generation of mutant cells with deletion of target genes.

The preparation method can be applied to human embryonic stem (ES) cells and other types of cells for similar purposes, which contributes to functional analyses of those mutant cells, mutant organoids, and mutant animals with deletion of target genes.

### BRIEF DESCRIPTION OF DRAWINGS

FIG. 1 shows a schematic diagram of the growth of ES cells before and after transfection and puromycin selection, where A shows that D1911 ES cells were of relatively high density and collected after trypsin digestion for transfection; B shows the growth of D1911 ES cells after 1 day of transfection; C shows that ES cells started to die rapidly after 1 day of puromycin selection; D shows that most of ES cells died after 2 days of puromycin selection; E shows that ES cells restored to rapid growth after removal of puromycin and then 1 day of culturing with normal ES cell medium; F shows that ES cells were of relatively high density again after removal of puromycin and then 2 days of culturing with normal ES cell medium.

FIG. 2 shows a schematic diagram of the growth of ES cell colonies before and after being plated and picked, where A shows that transfected and enriched D1911 ES cells after puromycin selection were serially diluted and plated onto a 10-cm dish plate seeded with SNL feeder cells; B shows that well separated ES cell colonies appeared a few days after being plated onto the 10-cm dish plate seeded with SNL feeder cells; C shows that single ES cell colonies were large enough for being picked individually; D shows that single ES cell colonies were plated onto a 24-well plate seeded with SNL feeder cells after being picked individually and separated by trypsin digestion, and each well was used for accommodating one single ES cell colony; E shows that ES cell colonies were rapidly growing on the SNL feeder cells seeded on the 24-well plate; and F shows that a portion of ES cells were ready for being harvested and frozen for storage, and genomic DNA samples derived from the portion of the ES cells of these ES clones were used for PCR amplification to screen for mutant ES clones.

FIG. 3 shows some identified candidate mutant ES clones with deletion of the Zfp445 gene derived from D1911 ES cells.

FIG. 4 shows some identified candidate mutant ES clones with deletion of the Zfp445 gene derived from TC1 ES cells.

### DETAILED DESCRIPTION OF THE INVENTION

The specific embodiments will be described below to illustrate the implementation of the present disclosure. Those skilled can easily understand other advantages and effects of the present disclosure according to the contents disclosed by the specification. The present disclosure can also be implemented or applied through other different specific embodiments. Various modifications or changes can also be made to all details in the specification based on different points of view and applications without departing from the spirit of the present disclosure.

Before the detailed description of the embodiments of the present disclosure, it needs to understand that the protection scope of the present disclosure is not limited to the specific exemplary embodiments described below. It should further understand that the specific terms used in the embodiments are just for the description of the present disclosure, rather than limiting the protection scope of the present disclosure. Unless otherwise stated, the terms, for example, “a”, “an”, “this” etc., in a singular form in the specification and claim of the present disclosure also indicate plural form.

When numerical ranges are given in the description, it should understand that, unless otherwise indicated herein, both endpoints of each numerical range and any number between the two endpoints may be selected for use. Unless otherwise defined, all technical and scientific terms used in this disclosure have the same meaning as understood by those skilled in the art from the prior art, and the present disclosure can also be implemented by any methods, devices, and materials of the prior art similar or equivalent to those described in the embodiments.

Unless otherwise stated, the laboratory procedures, tests, and production methods disclosed in the present disclosure are those well-known and commonly used in the field of molecular biology, biochemistry, chromatin biology, analytical chemistry, cell culture, recombinant DNA, and other routine techniques.

The present disclosure provides a preparation method for mutant cells with deletion of target genes, at least including the following steps:
step (1) transfecting a sgRNA group into same host cells, where each sgRNA group contains two sgRNAs that target different sites of a same target gene, and the number of the sgRNA group can be one or more than one. When the number of the sgRNA group is no less than one, each different sgRNA group targets one different target gene.
Step (2) enriching the host cells expressing one or more sgRNA groups.
Step (3) culturing the host cells obtained from step (2) to produce mutant cells with deletion of target genes.

In the present disclosure, the sgRNA group for transfection targets two different target sites of the same target gene, which leads to large deletion of the target gene.

The target site means a genomic DNA sequence that is complementary to the sequence of one sgRNA in each sgRNA group.

A distance between two target sites corresponding to two sgRNAs in each sgRNA group may be more than 100 bp, more than several kilobases (kb), or even longer. However, the efficiency may become lower for generation of mutant cells with deletion of target genes when the distance is more than a few kilobases. The distance could be even less than 100 bp, if another method, such as direct sequencing of the PCR product for the target gene derived from the genomic DNA samples of the picked ES clones, is used to distinguish the deletion mutant product from the wild-type product.

The distance between two target sites corresponding to two sgRNAs in the sgRNA group refers to the number of base pairs between two genomic DNA sequences that are complementary to sequences of two sgRNAs in the sgRNA group.

In an embodiment, the distance between two adjacent target sites of the same target gene corresponding to two sgRNAs may be in a range of 100 bp to 1000 bp. If two adjacent target sites are far away from each other (i.e., more than 1000 bp apart in distance), the efficiency may be lower. Therefore, more cell clones may be picked to screen those mutant cells with a large deletion of more than 1000 bp. Alternatively, the amount of the vector expressing drug-resistance gene may be reduced to enrich those transfected cells expressing one or more sgRNA groups, which facilitates to obtain mutant cells with deletion of target genes. The distance between two adjacent target sites of the same target gene may be greater than 100 bp, because it is easier to screen deletion mutations through PCR followed by gel electrophoresis, which contributes to distinguishing different bands on the gel corresponding to different product sizes of deletion mutant product and wild-type product. However, the distance between two target sites of two sgRNAs in the sgRNA group can also be smaller than 100 bp to obtain the desired deletion mutations, as long as there is a method (such as direct sequencing of the PCR product for the target gene derived from the genomic DNA samples of the picked ES clones) or any other method for identifying and confirming the mutant ES clones with the deletion mutations.

Optionally, the distance may be 100-900 bp, 100-800 bp, 100-700 bp, 100-600 bp, 100-500 bp, 100-400 bp, 100-300 bp, 100-200 bp; 200-900 bp, 200-800 bp, 200-700 bp, 200-600 bp, 200-500 bp, 200-400 bp, 200-300 bp; 300-900 bp, 300-800 bp, 300-700 bp, 300-600 bp, 300-500 bp, 300-400 bp; 400-900 bp, 400-800 bp, 400-700 bp, 400-600 bp, 400-500 bp; 500-900 bp, 500-800 bp, 500-700 bp, 500-600 bp; 600-900 bp, 600-800 bp, 600-700 bp; 700-900 bp, 700-800 bp, or 800-900 bp.

In step (1), the number of the sgRNA group may be greater than one.

If the number of the sgRNA group is more than one, then different sgRNA groups target different genes or genomic regions. A host cell may express multiple sgRNA groups, and two sgRNAs of each sgRNA group target two different target sites of the same target gene. Multiple sgRNA groups may target multiple different target genes to knock out multiple genes at the same time.

In one embodiment, a DNA fragment expressing a sgRNA is located on a vector.

Preferably, two sgRNAs in the sgRNA group may be located on two different vectors for the convenience of constructing the vector, i.e., one vector may only express one sgRNA in each sgRNA group. However, two sgRNAs in each sgRNA group may be expressed from the same vector if needed.

In an embodiment, the host cell may be embryonic stem (ES) cells.

Preferably, before step (2), the preparation method for mutant cells with deletion of target genes further includes: transfecting a drug-resistance gene on an expression vector into the host cells at the same time.

In an embodiment, in step (2), a drug is added for the enrichment of the co-transfected host cells to remove the un-transfected host cells that do not contain the drug-resistance gene.

Optionally, the duration period for drug selection to enrich the co-transfected host cells may be 2 days or 3 days.

In an embodiment, the drug-resistance gene is located on an expression vector.

Preferably, the sgRNAs and the drug-resistance gene are not located on the same vector for the ease of adjusting the amount of the vector expressing the drug-resistance gene, so as to facilitate the enrichment of co-transfected cells.

Preferably, the initial amount of the vector expressing the drug-resistance gene is less than that of the vector expressing each sgRNA during the transfection.

When there are multiple sgRNA groups, the initial amount of the vector expressing the drug-resistance gene is also less than that of the vector expressing each sgRNA.

The ratio of the initial amount of the vector expressing the drug-resistance gene to the initial amount of the vector expressing each sgRNA can be adjusted. If no mutant cells with deletion of target genes are obtained, the initial amount of the vector expressing the drug-resistance gene can be reduced, so as to facilitate the enrichment of co-transfected cells to obtain mutant cells with deletion of target genes more easily.

Optionally, the initial amount of the vector expressing the drug-resistance gene and the initial amount of the vector expressing each sgRNA may refer to quantity.

The ratio of the amount of the vector expressing the drug-resistance gene to the total amount of the vectors expressing all of the sgRNAs may be in a range of 1:20 to 1:4.

In an embodiment, two sgRNAs are employed to knock out one target gene, and the ratio of the initial amount of the vector expressing each sgRNA to the initial amount of the vector expressing the drug-resistance gene is 2:1.

In an embodiment, the initial amount of the vector expressing the drug-resistance gene is less than 0.8 μg.

For example, 0.8 μg of the vector expressing a puromycin-resistance gene is used. The initial amount of the vector expressing the puromycin-resistance gene may be adjusted to less than 0.8 μg according to the selection requirement, so that co-transfected cells carrying the vectors expressing all sgRNAs can be enriched.

In an embodiment, the drug is an antibiotic.

In an embodiment, the antibiotic is puromycin.

In an embodiment, in step (1), the transfection of the sgRNA group into the same host cells adopts a lipofectamine-mediated transfection.

Until now, there is no report about using drug selection for obtaining mutant cells with deletion of target genes. The drug selection used in the preparation method of the present disclosure removes most un-transfected cells, so that the remaining cells after 2-3 days of drug selection can be enriched for obtaining the transfected cells expressing one or more of sgRNAs.

As used herein, the term “vector” refers to a nucleic acid vehicle that allows the insertion of polynucleotides into itself. If the vector can be used to express the encoded protein corresponding to the inserted polynucleotides, it is usually called an expression vector. The vector can be introduced into a host cell through transformation, transduction, or transfection, so that the genetic material of the vector can be expressed inside the host cell. The vector is widely used by those skilled in the art, which includes but is not limited to, plasmid, phage, virus, cosmid, and all kinds of artificial chromosome (for example, yeast artificial chromosome (YAC), bacterial artificial chromosome (BAC), P1 artificial chromosome (PAC), etc. The phage here includes but is not limited to A phage and M13 phage. The virus here includes but is not limited to plant virus and animal virus. And the animal virus-derived vector, which is known as viral vector, includes but is not limited to, retrovirus (including lentivirus), adenovirus, adeno-associated virus (AAV), herpesvirus (for example, herpes simplex virus), poxvirus, baculovirus, papilloma virus, polyoma virus (for example, SV40), etc. A vector may contain multiple elements for controlling gene expression. The vector includes but is not limited to, a promoter, a transcription start site (TSS), an enhancer, a selection marker, and a reporter gene. The vector may also contain a replication initiation sequence.

As used herein, the term “host cell” refers to a cell in which allows the vector to be introduced. The host cell includes but is not limited to, prokaryotic cells, such as E. coli or bacillus; fungi, such as yeast or aspergillus; insect cells, such as S2 cells or Sf9 cells; or animal cells, such as fibroblast cells, CHO cells, COS cells, NSO cells, Hela cells, BHK cells, HEK 293 cells, mouse embryonic stem (ES) cells, human embryonic stem (huES) cells, mouse iPS cells or human iPS cells, etc.

### Embodiment 1

1. Transfection of ES Cells

Two plasmids, which express two different sgRNAs targeting two different target sites of the same target gene, are used in the present disclosure to generate a large deletion of the target gene. In this embodiment, taking the Zfp445 gene as an example, the Zfp445 gene in two different mouse ES cell lines (D1911 and TC1) had been successfully knocked out to generate expected mutant ES cells with deletion at Zfp445.

Preparation Before Transfection:

Designing the sgRNAs targeting Zfp445, sequences of each sgRNA in the sgRNA group are as follows,

sgRNA1:
sgRNA1 upstream primer:
(SEQ ID NO: 1)
5′-caccGAATAGGAATTTGTGACGTCC
sgRNA1 downstream primer:
(SEQ ID NO: 2)
5′-aaacGGACGTCACAAATTCCTATTC

sgRNA1 is expressed from a double-strand DNA fragment formed from the two annealed sgRNA1 primers, i.e., sgRNA1 upstream primer and sgRNA1 downstream primer. sgRNA1 is then cloned into the pX330-derived vector (purchased from Addgene) and transfected into ES cells for expression of sgRNA1.

The sequence of sgRNA1 is:
(SEQ ID NO: 3)
5′-GAATAGGAATTTGTGACGTCC
sgRNA2:
sgRNA2 upstream primer:
(SEQ ID NO: 4)
5′-caccGAGCTCAGCGCAATCTTTATC
sgRNA2 downstream primer:
(SEQ ID NO: 5)
5′-aaacGATAAAGATTGCGCTGAGCT

sgRNA2 is expressed from a double-strand DNA fragment formed from the two annealed sgRNA2 primers, i.e., sgRNA2 upstream primer and sgRNA2 downstream primer. sgRNA2 is then cloned into the pX330-derived vector (purchased from Addgene) and transfected into ES cells for expression of sgRNA2.

The sequence of sgRNA2 is:
(SEQ ID NO: 6)
5′-GAGCTCAGCGCAATCTTTATC

Two plasmids targeting Zfp445, which are the pX330-derived vectors expressing sgRNA1 and sgRNA2, respectively, can be obtained as described above.

The above-mentioned different DNA plasmids used for transfection, i.e., the pX330-derived vectors expressing sgRNA1 and sgRNA2, respectively, and the expression vectors carrying the puromycin-resistance gene, are mixed evenly at certain dosages, then these mixed DNA plasmids and expression vectors are added to transfection reagent mixture containing lipofectamine 2000 or lipofectamine 3000 to make sure each DNA plasmid and expression vector can be transfected into ES cells with the similar probability. In addition, compared with those DNA plasmids expressing the sgRNAs that target Zfp445, less amount of the expression vectors containing the puromycin-resistance gene are added for screening ES colonies that have undergone CRISPR/Cas9 editing.

The expression vectors with the puromycin-resistance gene can be derived from the pX334 vector (purchased from Addgene) or other vectors.

Reduction in the amount of expression vectors carrying puromycin-resistance gene can increase the ratio of survived cells carrying two pX330-derived vectors expressing sgRNA1 and sgRNA2, respectively, after puromycin selection.

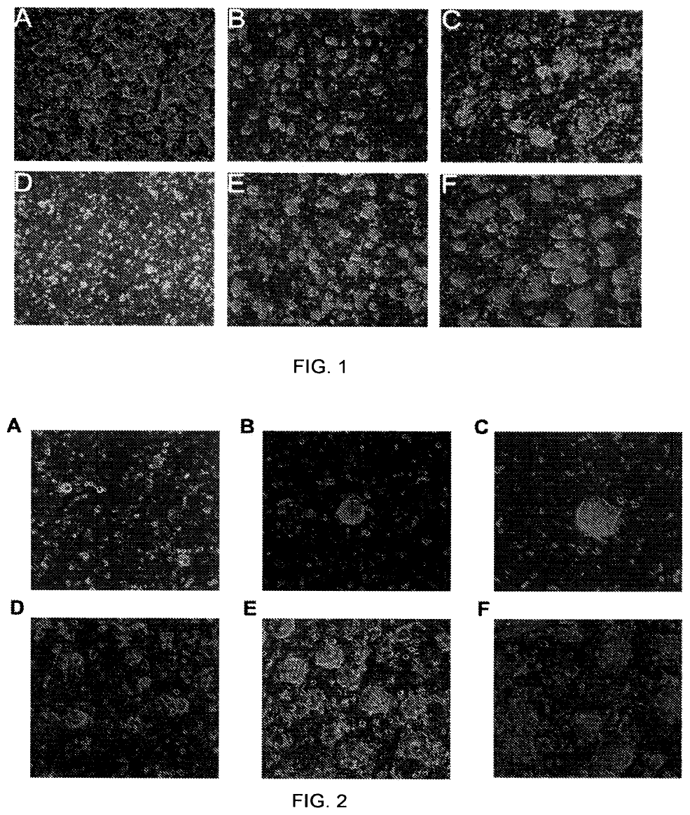

Before ES cell colonies are close to touching each other, i.e. before ES cells grow fully, they are digested with trypsin and then centrifuged and counted (FIG. 1A). Then feeder cells containing the puromycin-resistance gene are spread onto 6-well plates or 3.5-cm dish plates 4-12 hours before transfection, and later ES cells used for transfection are spread onto feeder cells containing the puromycin-resistance gene. The feeder cells refer to those prepared from mouse embryonic fibroblasts (MEF) carrying the puromycin-resistance gene.

Transfection Experiment:

(1) Two of 1.5-ml Eppendorf (EP) tubes were labeled as group 1 (lipofectamine group) and group 2 (DNA plasmid group), respectively. DMEM without serum and lipofectamine used for transfection were added to the 1.5-ml Eppendorf (EP) tube labeled as group 1, for example, 150 μl of DMEM and 6 μl of lipofectamine 2000 or lipofectamine 3000 were added. Specifically, lipofectamine was added to just below the liquid surface of DMEM by using a pipettor with a small range. The bottom of the EP tube was flicked several times with fingers or the EP tube was blown gently with the pipettor to realize even distribution. Then the EP tube was placed under room temperature for 5-10 minutes (min), afterward, DNA samples used for transfection were added, where the DNA samples included two pX330-derived vectors expressing sgRNA1 and sgRNA2, respectively, and the expression vectors expressing the puromycin-resistance gene.

(2) Group 2 is the DNA plasmid group. In this embodiment, two kinds of pX330-derived plasmids (including sgRNA1 and sgRNA2) are employed. In each transfection system, the amount of each kind of pX330-derived DNA plasmid targeting Zfp445 is 1.6 μg apiece, and the amount of the expression vectors with puromycin-resistance gene is 0.8 μg. These three kinds of plasmids are distributed evenly by blowing more than 15 times with the use of the small range pipettor. And the obtained plasmid mixture was added to an EP tube containing 150 μl of DMEM without serum.

(3) The obtained plasmid mixture was diluted and then added to the lipofectamine group, that is the solution in the EP tube labeled as group 2 was added to the solution in the EP tube labeled as group 1. The bottom of the EP tube was flicked several times or the EP tube was blown by the small range pipettor several times for mixing, and a re-mixing was performed 2 min later to acquire a more evenly distributed transfection mixture.

(4) The transfection mixture was placed at room temperature for 15 min. The medium on the plate with plated feeder cells which contains the puromycin-resistance gene are removed in advance, and approximately a half million of ES cells for transfection were spread onto the plated feeder cells, where the total amount of ES cells medium on the plate is controlled to be about 1 ml. The transfection mixture was added droplet to the plate used for transfection, and shaken horizontally to make sure a uniform distribution. The plate then was transferred to a typical cell culture incubator with the temperature of 37° C. and 5% of CO₂.

(5) About 6 hours later, more ES cell medium was added to the plate and the total amount of ES cell medium is controlled to be 3-4 ml per well. Afterward, the plate was placed in the cell culture incubator with the temperature of 37° C. and 5% of CO₂ again.

ES cell medium is prepared from DMEM cell culture medium containing glutamine, and the ES cell medium is further added with other ingredients at the final concentrations of 15% of fetal bovine serum (FBS), 1% of non-essential amino acids, 1% of penicillin and streptomycin, and a small amount of -mercaptoethanol (4 μl -mercaptoethanol per 500 ml of ES cell growth medium).

2. Enrichment of Transfected ES Cells

In order to enrich those cells that have been transfected successfully, puromycin selection was used to eliminate most of the cells which failed to be transfected.

(6) Drug selection (FIG. 1B) was performed one day (about 24 h) after transfection. The medium on the plate was removed, and 3-4 ml of ES cell medium containing 1 μg/ml of puromycin was added to the plate.

(7) Two days after transfection, the medium on the plate was still removed, and 3-4 ml of fresh ES cell medium containing 1 μg/ml of puromycin was added to the plate again (FIG. 1C). Since most of the cells which fail to be transfected are killed by medium containing puromycin, the operation of removing the previous day's medium and adding fresh medium can ensure the exclusion of most of the cells failing to be transfected.

(8) Three to four days after transfection, the medium containing puromycin was removed, and 2-3 ml of normal medium without puromycin was added. Generally, ES cells would grow rapidly after the replacement of the medium containing puromycin, and the single ES cell colonies can be observed under the microscope a couple of days later (FIG. 1E). Fresh normal medium without puromycin was added every day until the ES cell colonies grew to fill up the plate (FIG. 1F). When the ES cells were relatively sparse on the plate, 2-3 ml of fresh ES cell medium may be required per well, and when the ES cells were relatively full on the plate, 4-5 ml of fresh ES cell medium was needed for the growth of transfected cells.

3. Plating and Picking of ES Cells

After the transfected ES cells grew to fill up the plate, trypsin digestion was performed to harvest the transfected cells, the obtained cells were diluted and then spread onto the feeder cells at low density, so as to allow individual ES cell colonies to grow separately before being picked from the grown ES cell culture.

(9) Separating, Plating, and Culturing of Single ES Cell Colonies

(9.1) Feeder cells were spread on a 10-cm plate one day before the single ES cell colonies were ready for picking individually. The ES cell colonies nearly full of the plate were subject to trypsin digestion according to the standard cell culture method. The ES cell colonies undergone trypsin digestion were fully blown, and the number of harvested ES cells was counted afterward. The ES cells were then diluted according to the serial dilution method, and an appropriate number of the ES cells were spread onto the 10-cm plate containing the pre-plated feeder cells (FIG. 2A). Typically, 1×10³ ES cells were spread on a 10-cm plate, and the remaining ES cells were frozen in 1-2 of freezing vials as a backup.

(9.2) The medium was replaced one day after plating, and the amount of fresh ES cell medium was increased gradually in the following days until 20 ml of fresh ES cell medium was added to every 10-cm plate. Well-separated ES cell colonies grew on feeder cells after a few days until they were visible on the plate, and the formed ES cell colonies were large enough to allow easy picking of single ES cell colonies (FIG. 2C).

(10) Picking Individual ES Cell Colonies

Feeder cells were spread on a 24-well plate one day before picking ES cell colonies.

(10.1) Cell culture medium on a 10-cm culture plate was sipped up by using negative pressure devices, and the locations of visible ES cell colonies were circled on the outside surface of the bottom of the culture plate by using a marker. The ES cells were washed with 10 ml of sterilized 1×PBS solution carefully, and then the 1×PBS solution was sipped up. 10 ml of sterilized 1×PBS solution was added to the plate again and then the cell culture plate was placed under a microscope for picking, where the microscope was in a biosafety cabinet to prevent contamination. The dispersed and well-formed single ES cell colonies were selected under the microscopic view and transferred into different wells of the U-shaped 96-well plate.

(10.2) Trypsin digestion was usually performed after picking 12 ES cell colonies. The trypsin reagent and sterilized 1×PBS were homogeneously mixed in a centrifuge tube at a ratio of 1:1. And a drop of the homogeneously mixed solution was added to each well of the 96-well plate to perform trypsin digestion for 5 min. Then two drops of ES cell culture medium were added to each well to stop trypsin digestion. The culture plate was blown 15 times to disperse the picked ES cell colonies in each well by using the pipettor, while a multi-channel pipettor was used for pipetting multiple ES cell colonies simultaneously to save time. SNL feeder cells were spread on 24-well plates 1 day in advance, and 1 ml of ES cell culture medium was added to each well. The dispersed cells of each singly picked ES cell colony on the 96-well plate were transferred to each well of the 24-well plate seeded with SNL feeder cells one day earlier.

4. Cell Culture and Cryopreservation of the Picked ES Cell Colonies

Before collection and permanent cryopreservation, the cells of these picked ES cell colonies were expected to grow to be close to touching each other. In addition, genomic DNA samples were extracted from the cells of these ES cell colonies to perform PCR screening, so as to identify candidate mutant ES clones in the next steps.

(11) Replacement of cell culture medium was not required the next day. And fresh cell culture medium was added to each well of the 24-well plate every day thereafter.

(12) Daily replacement of cell culture medium was necessary from the third day until the cells in the wells grew to be close to touching each other (FIG. 2E-FIG. 2F).

(13) The cells of these singly picked ES cell colonies were temporarily cryopreserved, and candidate mutant ES clones were screened by PCR and then confirmed by sequencing.

(13.1) Cryopreservation of the ES cell colonies on the 24-well plates: the cell culture medium in the wells was sipped up, and each well of the plate was then washed once with 1 ml of sterilized 1×PBS. Afterward, the 1×PBS was sipped up, and 4 drops of a mixture of trypsin and PBS at a ratio of 1:1 were added to each well. The trypsin digestion was performed at room temperature for 5 minutes, and large cell clusters were blown apart by a sterile 1-ml pipettor.

(13.2) 1 ml of cryopreservation solution was added to each well of the 24-well plate to cryopreserve ES cell clones temporarily, and then ES cells in each well were blown 15 times with a 1-ml pipettor. Most of the resuspended ES cells in the cryopreservation solution were transferred to a labeled 2-ml freezing tube. The ES cells in the freezing tubes were then transferred to a −80° C. refrigerator or liquid nitrogen tank for temporary storage until the desired mutant ES cell clones were confirmed.

5. Identification of Mutant ES Cell Clones

Genomic DNA samples extracted from ES cell clones were used for PCR identification. PCR is generally performed by using primers spanning two CRISPR target sites to identify mutant ES clones carrying deletion mutations of the target genes. Sequencing of the PCR products of candidate mutant ES clones with the desired large fragment deletion was performed to identify the specific mutation caused by CRISPR.

(14) 1 ml of ES cell culture medium was added to the remaining ES cell suspension in each well of the 24-well plate in step 13 after cryopreservation, so as to extract genomic DNA samples from these ES cell colonies. The remaining ES cells of the cryopreserved ES cell colonies grew to be close to touching each other without the need to change the cell culture medium within a few days. Genomic DNA samples for genotype identification of ES cell clones were prepared according to the standard procedure for preparing genomic DNA from the adherent cell culture.

(15) PCR was employed to amplify the target sites with gene deletion from purified genomic DNA samples to identify candidate mutant ES clones. Specifically, two sgRNAs targeting two different positions of target genes were used to generate the deletion of relatively large genomic segments including some exons. These genomic DNA samples extracted from ES cell clones were subjected to PCR amplification followed by conventional DNA gel electrophoresis to identify mutant ES clones carrying deletion of the desired large fragment of the target gene.

(16) Sequencing of the PCR product with deletion of large or small DNA fragments of the target genes was performed to determine whether similar deletion occurred to both alleles of the target gene. The PCR product of mutant ES cell clones with the desired deletion of the target gene was purified and cloned into common bacterial vectors, such as pBluescript, to get a precise idea of the sequence deletion in the candidate ES clones.

The two primers below were utilized in PCR to screen mutant ES clones with deletion of the target gene Zfp445, and the sequences of the primers were as follows:

Zfp445-Forward,
(SEQ ID NO: 7)
5′-AGTGCGTCCTTCGTTACCTG
and
Zfp445-Reverse,
(SEQ ID NO: 8)
5′-GTGAAGGTAGCTGGGGATAC.

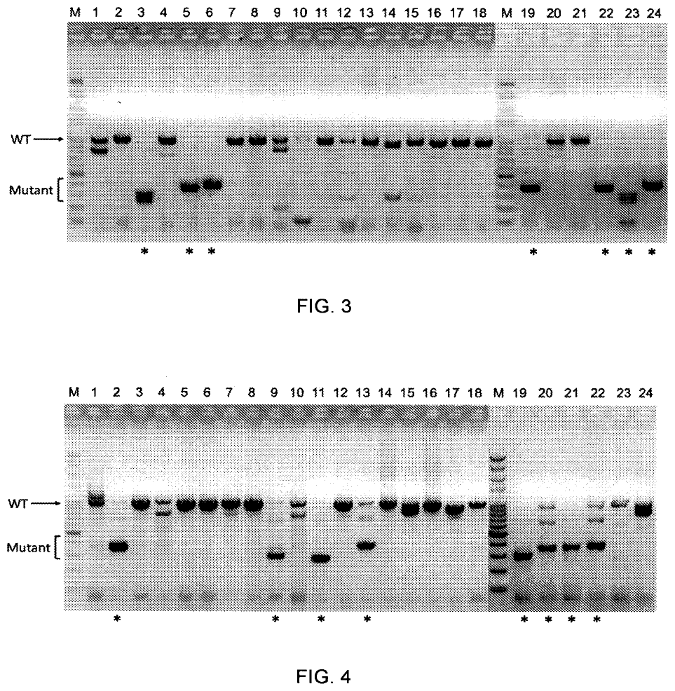

Sequencing was performed for a total of fifteen mutant ES clones with deletions shown in FIG. 3 and FIG. 4, and the expected deletions of the target gene Zfp445 were validated in the fifteen mutant ES clones.

As shown in FIG. 3, singly picked ES clones of transfected D1911 embryonic stem cells were cultured on the 24-well cell culture plate, and then genomic DNA samples of these ES clones were obtained. Two PCR primers spanning two sgRNA target sites of the Zfp445 gene were used for the PCR screening of these DNA samples. The distance between these two sgRNA target sites of the Zfp445 gene was about 765 bp, while the distance between these two PCR primers of the wild-type Zfp445 gene was 1039 bp. Seven of the screened twenty-four embryonic stem cell clones may contain a deletion of the Zfp445 gene fragment. Sequencing of the PCR products of these seven clones with deletion was performed, and deletion mutations of the target gene fragment induced by two sgRNAs occurred at the expected locations.

As shown in FIG. 4, genomic DNA samples were prepared from the individually picked ES cell colonies of transfected TC1 embryonic stem cells that were cultured on the wells of the 24-well cell culture plate. Two PCR primers spanning two sgRNA target sites of the Zfp445 gene were used for the PCR screening of these DNA samples. The distance between these two sgRNA target sites of the Zfp445 gene was about 765 bp, while the distance between these two PCR primers of the wild-type Zfp445 gene was 1039 bp. Eight of the screened twenty-four embryonic stem cell clones may contain a deletion of the Zfp445 gene fragment. Sequencing of the PCR products of these eight clones with deletion was performed, and deletion mutations of the target gene fragment induced by two sgRNAs occurred at the expected locations.

The success rate of obtaining mutant ES clones with deletions in the prior art is generally less than 1%. Sequencing of a total of fifteen mutant clones with deletions shown in FIG. 3 and FIG. 4 was performed and expected deletions of the target gene Zfp445 were verified in the present disclosure. The efficiency of obtaining mutant ES clones with deletions of the target gene Zfp445 in the present disclosure was 15/48=31.25%.

6. Amplification and Cryopreservation of Identified ES Cell Clone Samples

Feeder cells were spread on the 6-well cell culture plate and 2 ml of feeder cell culture medium was added to each well one day before thawing the mutant ES clones for expansion.

After PCR and sequencing, the mutant ES clones temporarily stored in a −80° C. refrigerator or liquid nitrogen tank in step 13 were thawed and then expanded on feeder cells. The ES clones in the cryopreservation tube were thawed, then the cryopreservation solution containing the cryopreserved cells was transferred to a 15-ml centrifuge tube added with 10 ml of ES cell culture medium, and further centrifuged at 1000 rpm for 5 min. The culture medium on the 6-well plate was sipped up, and 2 ml of ES cell culture medium was added to each centrifuge tube to resuspend the cell precipitate, and then transferred to the corresponding wells of the labeled 6-well plate.

Replacement of the culture medium was not required on the next day. From the third day, daily replacement of the culture medium was needed and the amount of ES cell culture medium gradually increased from 2 ml to 5 ml per well until the wells were full of ES cells, which can be frozen and stored for a long time.

If necessary, mutant ES clones with successful deletion could be further expanded on the feeder cells on the 10-cm cell culture plate, which is good for the cryopreservation of more tubes of mutant ES clones and the long-term storage of the mutant ES clones.

The above embodiments are intended to illustrate the implementations of the present disclosure and should not be construed as a limitation to the present disclosure. In addition, the various modifications listed herein and changes of methods and compositions in the present disclosure without departing from the scope and spirit of the present disclosure are obvious to those skilled in the art. Although the present disclosure has been described in combination with various specific embodiments of the present disclosure, it should be understood that the present disclosure should not be limited to these specific embodiments. In fact, various modifications as described above that are obvious to one skilled in the art should be included within the scope of the present disclosure.

Reference to Sequence Listing Submitted Electronically

This application incorporates by reference a Sequence Listing with this application as an XML file entitled “220490_ST26.XML” created on Mar. 8, 2023 and having a size of 12,288 bytes.

1. A preparation method for mutant cells with deletion of target genes, at least comprising the following:
step (1) transfecting a sgRNA group into same host cells, where the sgRNA group includes two sgRNAs that target two different sites of a same target gene, and the number of the sgRNA group is no less than one;
when the number of the sgRNA group is more than one, each different sgRNA group targets one different target gene;
step (2) enriching the host cells expressing one or more sgRNA groups;
step (3) culturing the host cells obtained from step (2) to produce mutant cells with deletion of target genes.

2. The preparation method for mutant cells with deletion of target genes according to claim 1, wherein the two sgRNAs in the sgRNA group target the same target gene.

3. The preparation method for mutant cells with deletion of target genes according to claim 1, wherein the following step is included before the step (2): transfecting a drug-resistance gene to the host cells as described in the step (1).

4. The preparation method for mutant cells with deletion of target genes according to claim 3, wherein the enriching the host cells expressing one or more sgRNA groups in the step (2) is employing a drug to remove the host cells that do not contain the drug-resistance gene.

5. The preparation method for mutant cells with deletion of target genes according to claim 3, wherein the drug is selected from antibiotics.

6. The preparation method for mutant cells with deletion of target genes according to claim 3, wherein each sgRNA and the drug-resistance gene are included on expression vectors.

7. The preparation method for mutant cells with deletion of target genes according to claim 6, wherein the sgRNA and the drug-resistance gene are included on different expression vectors.

8. The preparation method for mutant cells with deletion of target genes according to claim 7, wherein when the number of the sgRNA group is one, an initial amount of the vector containing the drug-resistance gene is less than an initial amount of the vector containing each sgRNA in the sgRNA group during transfection;
when the number of the sgRNA group is more than one, an initial amount of the vector containing the drug-resistance gene is less than an initial amount of the vector containing each sgRNA during transfection.

9. A mutant cell with deletion of target gene prepared from the preparation method according to claim 1.
